(19) United States
(12) Patent Application Publication
(10) Pub. No.: US 2023/0240992 A9
(43) Pub. Date: Aug. 3, 2023

(54) COMPOSITIONS AND METHODS FOR TOPICAL DELIVERY

(71) Applicant: Ziropa, Inc., Sunnyvale, CA (US)

(72) Inventor: Jacob Waugh, Irvine, CA (US)

(21) Appl. No.: 17/444,829

(22) Filed: Aug. 10, 2021

Related U.S. Application Data
(60) Provisional application No. 63/063,755, filed on Aug. 10, 2020.

(51) Int. Cl. A61K 9/14; A61K 31/197; A61K 31/195; A61K 31/662

(52) U.S. Cl. CPC A61K 9/14; A61K 31/197; A61K 31/195; A61K 31/662

(57) ABSTRACT

Compositions comprising an active agent and a decoy molecule and methods for treating pain by topically administering such compositions are described herein. The compositions may be powdered. Compositions include a decoy molecule with a specified average molecular weight and no high molecular weight decoy molecule. The extracellular components include hyaluronic acid, collagen, fibronectin, elastin, lectin, and fragments thereof and combinations thereof.

### CROSS-REFERENCE TO RELATED APPLICATIONS

This application claims priority from U.S. Provisional No. 63/063,755, entitled “Compositions and Methods for Topical Delivery” filed Aug. 10, 2020, the contents of which are hereby incorporated by reference in their entirety.

### GOVERNMENT INTERESTS: NOT APPLICABLE

### PARTIES TO A JOINT RESEARCH AGREEMENT: NOT APPLICABLE

### INCORPORATION OF MATERIAL ON COMPACT DISC: NOT APPLICABLE

### BACKGROUND

The current standard of practice for the non-invasive treatment of the various neuropathies include the following drug classes: (1) The Non Steroidal Anti-inflammatory Drugs (NSAIDs); (2) Narcotic analgesics; (3) Tricyclic antidepressants; and (4) Anticonvulsants. These classes are available commercially as oral products and, generally, are administered orally. Because they are distributed systemically throughout the body, various side effects limit their effectiveness. The NSAIDs, for example, when taken orally cause gastric distress—including ulcers. Narcotic analgesics, such as morphine, are sparingly prescribed for chronic pain because of the well-known addictive and central nervous system (CNS) side effects and gastrointestinal side effects resulting from their single administration. These classes of drugs share other common side effects, including drowsiness, dizziness, disorientation, and gastrointestinal upset among others.

### SUMMARY OF THE INVENTION

Embodiments of the invention are directed to compositions containing an effective amount of a powdered active agent. Other embodiments include a composition including a powdered active agent and a powdered penetration enhancer.

In some embodiments, the penetration enhancer may be selected from alcohols, glycols, fatty acids, fatty esters, fatty ethers, occlusive agents, surface active agents, dimethyl amino propionic acid derivatives, terpenes, decoy molecules, sulfoxides, cyclic ethers, amides, amines, and the like and combinations thereof, and in some embodiments, the penetration enhancer may be hyaluronic acid, collagen, fibronectin, elastin, lectin, fragments thereof, and the like and combinations thereof. In particular embodiments, the penetration enhancer may be dehydrated or lyophilized, and in some embodiments, the penetration enhancer may have an average molecular weight of about 2,000 Da to about 100,000 Da. The penetration enhancer of embodiments may be any of chemical permeation enhancers (CPEs), non-ionizable glycol ethers, known peptides or protein fragments, microparticles or nanoparticles, and combinations thereof.

In various embodiments, the powdered active agent and powdered penetration enhancer may each individually have an average particle size of about 0.01 μm to about 100 μm. In some embodiments, the active agent may be baclofen, vigabatrin, gabapentin, pregabalin, y-amino-phosphinic acid derivatives, and combinations thereof. In certain embodiments, the composition may be substantially free of water. In various embodiments, the composition may include about 1 wt. % to about 95 wt. % active agent, and in some embodiments, the composition may include about 0.1 wt. % to about 10 wt. % penetration enhancer.

In some embodiments, the composition may further include a stabilizer such as, for example, alkanols, disodium EDTA (ethylenediamine tetraacetate), EDTA salts, EDTA fatty acid conjugates, isothiazolinone, parabens, methylparaben and propylparaben, propylene glycols, sorbates, urea derivatives, diazolidinyl urea, and combinations thereof. In some embodiments, the composition may further include a carrier, excipient, diluent, filler, disintegrant, desiccant, binder, lubricant, surfactant, hydrophobic vehicle, water soluble vehicle, emulsifier, buffer, humectant, moisturizer, solubilizer, preservative, colorant, plasticizer, or combinations thereof.

Further embodiments include methods for treating a subject in need treatment including the steps of topically administering to a surface tissue of the subject a composition comprising a powdered active agent, and a powdered penetration enhancer.

In some embodiments, the penetration enhancer may be selected from alcohols, glycols, fatty acids, fatty esters, fatty ethers, occlusive agents, surface active agents, dimethyl amino propionic acid derivatives, terpenes, decoy molecules, sulfoxides, cyclic ethers, amides, amines, and the like and combinations thereof, and in some embodiments, the penetration enhancer may be hyaluronic acid, collagen, fibronectin, elastin, lectin, fragments thereof, and the like and combinations thereof. In particular embodiments, the penetration enhancer may be dehydrated or lyophilized, and in some embodiments, the penetration enhancer may have an average molecular weight of about 2,000 Da to about 100,000 Da. The penetration enhancer of embodiments may be any of chemical permeation enhancers (CPEs), non-ionizable glycol ethers, known peptides or protein fragments, microparticles or nanoparticles, and combinations thereof.

In various embodiments, the powdered active agent and powdered penetration enhancer may each individually have an average particle size of about 0.01 μm to about 100 μm. In some embodiments, the active agent may be baclofen, vigabatrin, gabapentin, pregabalin, γ-amino-phosphinic acid derivatives, and combinations thereof. In certain embodiments, the composition may be substantially free of water. In various embodiments, the composition may include about 1 wt. % to about 95 wt. % active agent, and in some embodiments, the composition may include about 0.1 wt. % to about 10 wt. % penetration enhancer.

In some embodiments, the composition may further include a stabilizer such as, for example, alkanols, disodium EDTA (ethylenediamine tetraacetate), EDTA salts, EDTA fatty acid conjugates, isothiazolinone, parabens, methylparaben and propylparaben, propylene glycols, sorbates, urea derivatives, diazolidinyl urea, and combinations thereof. In some embodiments, the composition may further include a carrier, excipient, diluent, filler, disintegrant, desiccant, binder, lubricant, surfactant, hydrophobic vehicle, water soluble vehicle, emulsifier, buffer, humectant, moisturizer, solubilizer, preservative, colorant, plasticizer, or combinations thereof.

### DESCRIPTION OF THE DRAWINGS: NOT APPLICABLE

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but are not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entirety are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to pharmaceutical compositions containing an effective amount of an active agent in powdered form for topical pain management. In some embodiments, such pharmaceutical compositions may include a powdered penetration enhancer or permeation enhancer such as, for example, decoy molecules. The powdered active agent and powdered penetration enhancer can be admixed to create the pharmaceutical compositions embodiments. In some embodiments, such admixtures may include no further components. Such embodiments include compositions that are non-toxic to humans or animals, non-irritating in humans or animals, and non-sensitizing in humans and animals. In certain embodiments, the permeation or penetration enhancer may include a decoy molecule.

The active agents used in the pharmaceutical compositions and methods of the invention are not limited. For example, the active agent can be hydrophilic, lipophilic, amphiphilic or hydrophobic, and, in some embodiments, the active agent can be solubilized, dispersed, or partially solubilized and dispersed, in an encapsulation coating. Such active agents can be any compound or mixture of compounds having therapeutic or other value when administered to an animal, particularly to a mammal, such as drugs, nutrients, cosmeceuticals, diagnostic agents, nutritional agents, and the like. It should be appreciated that the categorization of an active ingredient as hydrophilic or hydrophobic may change, depending upon the particular salts, isomers, analogs and derivatives used

In some embodiments, the active agent may be hydrophobic. Hydrophobic active ingredients are compounds with little or no water solubility. Intrinsic water solubilities, i.e. water solubility of the unionized form, for hydrophobic active agents are generally less than about 1% by weight, and typically less than about 0.1% or 0.01% by weight. In particular embodiments, the active agent may be a hydrophobic drug.

Suitable hydrophobic active agents include, for example, analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malariale, anti-migrainc agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and the like and combinations and mixtures thereof.

Non-limiting examples of suitable hydrophobic active agents include acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, and the like and combinations thereof, including isomers and derivatives of such hydrophobic active agents.

In some embodiments, the active agent may be an anticonvulsant. Examples of useful anticonvulsants include, but are not limited to, acetylpheneturide, albutoin, aloxidone, aminoglutethimide, 4-amino-3-hydroxybutyric acid, atrolactamide, beclamide, buramate, calcium bromide, carbamazepine, cinromide, clomethiazole, clobazam, clonazepam, decimemide, diethadione, dimethadione, doxenitroin, eterobarb, ethadione, ethosuximide, ethotoin, felbamate, fluoresone, fosphenyloin, gabapentin, 5-hydroxytryptophan, lamotrigine, levetiracetam, magnesium bromide, magnesium sulfate, mephenyloin, mephobarbital, metharbital, methetoin, methsuximide, 5-methyl-5-(3 -phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenyloin, phethenylate sodium, potassium bromide, pregabalin, primidone, progabide, sodium bromide, sodium valproate, solanum, strontium bromide, suclofenide, sulthiame, tetrantoin, tiagabine, topiramate, trimethadione, valproate semisodium, valproic acid, valpromide, vigabatrin, and zonisamide, and the like and derivatives and combinations thereof.

In certain embodiments, the active agent may be a GABA analog, such as, for example, baclofen, vigabatrin, gabapentin, pregabalin, or a γ-amino-phosphinic acid derivative. Gamma-aminobutyric acid (GABA) is one of the inhibitory neurotransmitters in the central nervous system of mammals. It acts at inhibitory synapses in the brain by binding to specific transmembrane receptors in the plasma membrane of both pre- and postsynaptic neuronal processes. This binding causes the opening of ion channels to allow the flow of either negatively charged chloride ions into the cell or positively charged potassium ions out of the cell. This action results in a negative change in the transmembrane potential, usually resulting in hyper-polarization. Three general GABA receptors have been identified, GABAA, GABAB and GABAC. GABAA and GABAC are ionotropic receptors, whereas GABAB is a G protein coupled metabotropic receptor. Low levels of GABA have been linked to many diseases including but not limited to epileptic seizures, multiple sclerosis, action tremors, panic, anxiety, and depression

A number of GABA analogs have been identified. These GABA analogs include but are not limited to baclofen, vigabatrin, gabapentin, and pregabalin. Unlike GABA itself, these GABA derivatives can pass through the blood-brain barrier likely by active transport mechanism, and demonstrate pharmaceutical utility clinically. Baclofen is a GABAB agonist that is clinically used for the treatment of spastic movement or relief of pain, especially in instances of spinal cord injury, spastic diplegia, multiple sclerosis and trigeminal neuralgia. Vigabatrin is an anticonvulsant that has been used as an adjunctive treatment for diseases such as epilepsy and complex partial seizures. Gabapentin and pregabalin were originally developed for the treatment of epilepsy, but have been proven to be effective to relieve chronic pain, especially neuropathic pain such as diabetic neuropathic pain and postherpetic neuralgia. Gabapentin and pregabalin have also shown effectiveness in fibromyalgia treatment.

Studies suggest that the neuropathic pain alleviation action is mediated through alpha-2-delta subunit of the voltage-gated N-type calcium ion channel, a high affinity binding site in neuronal membranes. This subunit has been implicated in the maintenance of mechanical hypersensitivity in models of neuropathic pain. Calcium ions enable vesicles containing neurotransmitters to fuse with the presynaptic membrane, an action that promotes the release of neurotransmitters that inhibit the synaptic cleft. Both gabapentin and pregabalin probably exert their therapeutic effects by blocking calcium influx via alpha-2-delta receptors and reducing the release of neurotransmitters that transmit nociceptive signals between neurons. In vitro findings suggest that gabapentin may reduce presynaptic release of excitatory neurotransmitters such as glutamate and norepinephrine.

The active agent is such embodiments may be in powdered form, meaning the powders are solid in a dry, finely divided state. Such powders may have an average particle size of about 0.01 μm to about 100 μm, about 0.05 μm to about 50 μm, about 0.1 μm to about 10 μm or any range or individual particle size encompassed by these example ranges. In some embodiments, the powders may be “dry,” meaning the powdered active agent is “substantially free” of water. “Substantially free,” as used herein means containing less than about 2%, less than about 1%, less than about 0.5%, less than about 0.1%, less than about 0.05%, less than about 0.01% by weight water based on the total weight of the composition or any concentration encompassed by these example ranges. In certain embodiments, “substantially free” means completely free of water, i.e. containing no, or an immeasurable amount of water.

In some embodiments, the compositions may consist of the powdered active agent alone. Such compositions may contain no additional biologically active components such as penetration enhancers, but may include fillers, diluents, and excipients that may aid in stabilization, handling, or application of the compositions to subjects in need of treatment. Examples of powdered fillers, diluents, and excipients include, but are not limited to, starches such as corn starch and potato starch; cellulose and its derivatives such as sodium carboxymethyl cellulose, ethyl cellulose and cellulose acetate; powdered tragacanth; malt; gelatin; talc, bentonite, insoluble salts of bismuth, boric acid, calcium carbonate, magnesium stearate, talc, titanium dioxide, zinc oxide, zinc stearate, and the like and combinations thereof. The fillers, diluents, and excipients may also act as desiccants, which may improve the shelf life of the compositions. In certain embodiments, the compositions may include powdered preservatives such as alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolidinyl urea, and the like and combinations thereof.

In some embodiments, the compositions may include a penetration or permeation enhancer. The penetration or permeation enhancer may be any penetration or permeation enhancer known in the art. For example, in various embodiments, the penetration enhancer may be alcohols, glycols, fatty acids, fatty esters, fatty ethers, occlusive agents, surface active agents, dimethyl amino propionic acid derivatives, terpenes, decoy molecules, sulfoxides, cyclic ethers, amides, amines, and the like and combinations thereof. In certain embodiments, the penetration enhancer may be a powder, which may be “substantially free” from water. The powdered penetration or permeation enhancers may be existing powders, liquids, or water containing solids that have been treated to remove any water associated with the penetration or permeation enhancer. For example, in some embodiments, the permeation enhancer may be a lyophilized, freeze dried, crystalized, or dehydrated powder. Removing water from compositions by these methods is well known in the art.

In some embodiments, the penetration or permeation enhancer may be a decoy molecule, and the pharmaceutical compositions may contain an effective amount of an active agent and a decoy molecule. “Decoy molecules” are known in the art and are generally composed of an extracellular matrix component or fragments thereof and/or combinations thereof that is capable of causing rearrangement of tissues that the composition contacts by temporarily disrupting cell-cell (i.e. intercellular) and cell-scaffold attachment allowing the active agent to pass through cell layers and passive intracellular crossing of the active agent into cells throughout the tissue. Thus, decoy molecules act as permeation or penetration enhancers. Without wishing to be bound by theory, the selection of the decoy molecule(s), the average molecular weight, the presence (or absence) of high molecular weight decoy molecule, the amount of decoy molecule, the botulinum toxin agent being delivered and the target surface tissue will affect the ability of the botulinum toxin to be delivered topically to the desired site of action. For example, surface tissue that contains significant glandular structures or hair follicles (or hair) may utilize decoy molecules of intermediate molecular weight (such as about 20,000 Da to about 40,000 Da) or if a more diffuse effect is desired, decoy molecules of lower molecular weight (such as less than 15,000 Da) may be utilized.

The decoy molecule of various embodiments may be composed of any extracellular matrix component, including hyaluronic acid, collagen, fibronectin, elastin, lectin, and combinations thereof or hyaluronic acid fragments, collagen fragments, fibronectin fragments, elastin fragments, lectin fragments, and combinations thereof. The type and size of the decoy molecule may impact the depth of penetration of the active agent and can be specifically selected to provide delivery of the active agent to specific tissues.

In some embodiments, the decoy molecule may be hyaluronic acid or fragments of hyaluronic acid. Hyaluronic acid is a polymer of disaccharides that is known to interact with, for example, CD44, receptor for hyaluronic acid-mediated motility (RHAMM), and intercellular adhesion molecule-1 (ICAM-1). CD44 is widely distributed throughout the body and mediates cell interaction with hyaluronic acid. ICAM-1 is a metabolic cell surface receptor for hyaluronic acid, and binding of hyaluronic acid to ICAM-1 may contribute to the control of ICAM-1-mediated inflammatory activation.

In some embodiments, the decoy molecule may be collagen or fragments of collagen. The collagen used in such embodiments can be isolated in any form and source and includes collagen type I, collagen type II, collagen type III, collagen type IV, and collagen type V or combinations of these collagens or fragments thereof. Collagen decoy molecules can be in any form including fibrillary, non-fibrillary, disorganized collagen, and combinations and fragments thereof. In some embodiments, collagen decoy molecules can be made by, for example, hydrolysis of full length or large fragments of collagen.

In some embodiments, the decoy molecule may be fibronectin or fragments of fibronectin. Fibronectin is a protein dimer, consisting of two nearly identical monomers linked by a pair of disulfide bonds. Fibronectin binds to membrane-spanning receptor proteins called integrins and collagen, fibrin, heparin sulfate, proteoglycans and other extracellular matrix components. Fibronectin is a large glycoprotein. Fibronectin decoy molecules may therefore include fragments of fibronectin.

In some embodiments, the decoy molecule may be elastin or a fragment of elastin. Elastin is a protein found in connective tissue and allows many tissues in the body to resume their shape after stretching or contracting. Elastin fragments may be obtained commercially or may be generated by protease digestion, such as using proteinase K or thermolysin. In embodiments, the elastin or elastin fragments may be Elastin E91 (from Protein Preparations, Inc., St. Louis, Mo.), ProK, ProK-60 and/or ProK-60P, which are elastin peptide mixtures derived from the proteolytic digestion of insoluble elastin derived from bovine neck ligaments. In embodiments, the elastin or elastin fragments may comprise an amino acid sequence selected from the group consisting of: GAAPG, GVVPG, GGGPG, GLLPG, GIIPG, GSSPG, GTTPG, GCCPG, GMMPG, GFFPG, GYYPG, GWWPG, GDDPG, GNNPG, GEEPG, GQQPG, GRRPG, GHHPG, GKKPG, GPPPG, G3Hyp3HypPG (Glycine-3-hydroxyproline-3-hydroxyproline-Proline-Glycine), G4Hyp4HypPG (Glycine-4-hydroxyproline-4-hydroxyproline-Proline-Glycine), RRPEV, QPSQPGGV, PGGV, GPGV, KPGV, GPGL, EGSA, PGGF, GGGA, KPGKV, PGGV, KPKA, GPGGV, GPQA, GGPGI, PGPGA, GPGGV, GQPF, GGKPPKPF, GGQQPGL, GGPGI, VGVAPG, IGVAPG, PGGVLPG, VGVVPG, IGLGPGGV, VGAMPG, VGLSPG, IGAMPG, IGLSPG, GVAPGV, VAPGVG, APGVGV, PGVGVA, GVGVAP and combinations thereof.

In embodiments, the decoy molecule, including various extracellular matrix components, extracellular matrix component fragments, and combinations thereof, may have specified average molecular weight. For example, in some embodiments, the decoy molecule may have an average molecular weight of about 2,000 Da to about 100,000 Da, about 2,000 Da to about 60,000 Da, about 2,000 Da to about 50,000 Da, about 2,000 Da to about 40,000 Da, about 2,000 Da to about 30,000 Da, about 2,000 to about 20,000 Da, about 2,000 to about 15,000 Da, about 2,000 Da to about 10,000 Da, about 5,000 Da to about 40,000 Da, less than about 60,000 Da, less than about 50,000 Da, less than about 40,000 Da, less than about 30,000 Da, less than about 20,000 Da, less than about 15,000 Da, less than about 10,000 Da, less than about 5,000 Da, about 60,000 Da, about 50,000 Da, about 40,000 Da, about 30,000 Da, about 20,000 Da, about 15,000 Da, about 12,500 Da, about 10,000 Da, about 8,500 Da, about 7,500 Da, about 5,000 Da, about 2,000 Da to about 5,000 Da, about 5,000 Da to about 10,000 Da, about 10,000 Da to about 20,000 Da, about 20,000 Da to about 30,000 Da, about 30,000 Da to about 40,000 Da, about 20,000 Da to about 40,000 Da, about 40,000 Da to about 60,000 Da, or about 60,000 Da to about 100,000 Da or any range or individual average molecular weight falling within these example ranges. In certain embodiments, the decoy molecule have a molecular weight above 150,000 Da, less than 125,000 Da, less than 100,000 Da, less than 90,000 Da, less than 80,000 Da, less than 70,000 Da, less than 60,000 Da, less than 55,000 Da, less than 50,000 Da, less than 45,000 Da, less than 40,000 Da, or less than 35,000 Da.

In some embodiments, the decoy molecule may be dehydrated or lyophilized. Lyophilization (freeze-drying) is a low temperature dehydration process that involves freezing a product, lowering pressure, then removing ice in the product by sublimation. In contrast, dehydration processes use heat to drive off water by evaporation. Lyophilization typically results in a high quality product because the low temperature used in processing doesn't disrupt the secondary and/or tertiary structural elements of protein molecules, and in some embodiments, is the preferred means for creating decoy molecule powders. In addition, lyophilization may increase the shelf life of the products, making the compositions of embodiments easy to store, ship, and later reconstitute if necessary.

In some embodiments, the decoy molecules may spray dried. Spray drying is a single step process in which protein solutions are sprayed into a drying chamber where droplets are rapidly dried in a hot drying medium. Dehydrated particles are collected using, for example, a cyclone and typically have uniform sizes and morphologies. The spray drying procedure is fast and is effective at stabilizing proteins for incorporation into the compositions of the invention.

In some embodiments, the decoy molecules may be spray freeze dried. The spray freeze drying includes spraying a protein solution into a freezing chamber in which droplets of the protein solution are rapidly frozen. The frozen droplets are then dehydrated by sublimation and the dehydrated particles are collected. As with spray drying, spray freeze dried particles typically are uniform in size and in many cases the particles are spherical. The spray freeze drying procedure is fast and is effective at stabilizing proteins for incorporation into the compositions of the invention.

In some embodiments, the decoy molecules may be turned into a powder by supercritical fluid drying. In the supercritical fluid drying process a protein solution is combined with a volatile solvent or gas (“supercritical medium”) that is at critical temperature and pressure. The supercritical medium may vary among embodiments and may be, for example, carbon dioxide, nitrous oxide, ammonia, or organic hydrocarbons such as methane, ethane, ethane, propane, propene, n-butane, i-butane and n-pentane; alcohols such as benzyl alcohol, methanol, ethanol, (iso)propanol, (iso)butanol, halogenated compounds such as chlorotrifluoromethane, monofluoro methane; cyclic hydrocarbons such as toluene, pyridine, cyclohexane, cyclohexanol and o-xylene. Supercritical points for these supercritical media are known in the art. For example, a protein solution that is dried by supercritical fluid drying in carbon dioxide (CO₂) is carried out at a temperature of the drying vessel of about 60° C. Because the proteins in the solution are not subjected to potentially harsh temperature changes, the supercritical fluid drying method may result in higher activity of reconstituted particles. Thus, powdered decoy molecules produced by the supercritical fluid drying process can be incorporated into the compositions of the invention.

Decoy molecules may facilitate delivery of the active agent more efficiently than penetration enhancers that merely act on the stratum corneum of the skin. That is, the amount of active agent delivered at the site of administration is much more when compared to the delivery of the active agent without decoy molecules or active agents in compositions using other penetration or permeation enhancers. In some embodiments, the compositions may provide therapeutic equivalence of known topically administered the active agent with that an administered dose that is equal to or at least about 75% less than a standard dose, equal to or about 50% less than a standard dose, equal to or about 25% less than a standard dose, equal to or about 10% less than a standard dose, about 1.0% to about 75% less than a standard dose, about 1.0% to about 50% less than a standard dose, about 1.0% to about 25% less than a standard dose, about 1.0% to about 10% less than a standard dose, about 2.0% to about 75% less than a standard dose, about 2.0% to about 50% less than a standard dose, about 2.0% to about 25% less than a standard dose, about 2.0% to about 10% less than a standard dose, or any range or individual value encompassed by these example ranges.

The compositions of embodiments may contain an effective amount of an active agent and an effective amount of a decoy molecule. An effective amount of an active agent may vary among embodiments and may depend on the active agent incorporated into the composition, the activity of the active agent, the disease being treated, and the location and size of the treatment area. In various embodiments, the amount of active agent may be about 1 wt. % to about 95 wt. %, about 1 wt. % to about 75 wt. %, about 1 wt. % to about 50 wt. %, about 1 wt. % to about 40 wt. %, about 1 wt. % to about 30 wt. %, about 1 wt. % to about 25 wt. %, about 1 wt. % to about 10 wt. %, or any individual amount or any ranges between any two of these values. Weight percent is based on the total weight of the composition. including all its components.

In some embodiments, the decoy molecule may make up the remainder of the composition and can be present in amounts including, for example, about 5 wt. % to about 90 wt. %, about 5 wt. % to about 75 wt. %, about 10 wt. % to about 70 wt. %, about 10 wt. % to about 60 wt. %, about 10 wt. % to about 50 wt. %, about 10 wt. % to about 25 wt. %, or any range or individual concentration falling within these example ranges. Weight percent is based on the total weight of the composition. including all its components.

In other embodiments, the composition may include an active agent in the concentrations described above, a decoy molecule, and various fillers, diluents, carriers, excipients, desiccants, preservatives, additives, and combinations thereof. In such embodiments, the decoy molecule may be present in a concentration of about 0.1 wt. % to about 10 wt. %, about 0.1 wt. % to about 9 wt. %, about 0.1 wt. % to about 8 wt. %, about 0.1 wt. % to about 7 wt. %, about 0.1 wt. % to about 6 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, about 0.1 wt. %, about 0.5 wt. %, about 0.25 wt. % to about 10 wt. %, about 0.25 wt. % to about 1.0 wt. %, about 0.25 wt % to about 1.5 wt %, about 0.25 wt. % to about 2.0 wt. %, about 0.25 wt. % to about 3.0 wt. %, about 0.5 wt. % to about 5.0 wt. %, about 0.5 wt. % to about 3.0 wt. %, about 0.75 wt. % to about 7.5 wt. %, about 1.0 wt % to about 3 wt %, about 1.0 wt. % to about 5.0 wt. %, about 1 wt. %, about 2 wt. %, about 5 wt. %, about 10 wt. %, or any range or individual concentration falling within these example ranges. Weight percent is based on the total weight of the composition. including all its components. The mass of the decoy molecule in such embodiments can vary between about 1 microgram to about 100 milligrams.

In some embodiments, the compositions may further include hyaluronidase, elastase enzymes, or combinations thereof. The hyaluronidase enzyme family consists of enzymes capable of hydrolyzing or “breaking down” the polysaccharide hyaluronic acid. Hyaluronic acid is an important constituent of connective tissue. Thus, hyaluronidases, which can spread and diffuse rapidly through tissues, can modify the permeability and viscosity of the intercellular cement by hydrolyzing hyaluronic acid. Hyaluronidases can be broadly classified into three groups: mammalian-type hyaluronidases (EC 3.2.1.35) are endo-beta-N-acetylhexosaminidases that produce tetrasaccharides and hexasaccharides as the major end products. They have both hydrolytic and transglycosidase activities, and can degrade hyaluronan and chondroitin sulfates (CS), specifically C4-S and C6-S. Bacterial hyaluronidases (EC 4.2.99.1) degrade hyaluronan and, and to various extents, CS and DS. They are endo-beta-N-acetylhexosaminidases that operate by a beta elimination reaction that yields primarily disaccharide end products. Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

The hyaluronidase disclosed herein can be derived from any source. For example, hyaluronidase may be recovered from bovine protein (bovine type), leeches or bacteria (e.g. in the form of hyaluronate lyase, vegetables, or genetically modified bacteria, yeast, or cloned mammalian or human cells. Hyaluronidase can be also obtained commercially, from, for example, Wyeth-Ayerst (Wydase®), Abbot (Hyazyme®), Bristol-Myers Squibb (Enzodase®), and Ortho Pharmaceuticals (Diffusin®). Non-limiting examples of hyaluronidases that can be used in the compositions are human hyaluronidase-1, human hyaluronidase-2, human hyaluronidase-3, human hyaluronidase-4, and human PH2O.

Elastase (EC 3.4.21.36.) is a member of a group of enzymes termed “serine proteases,” which are characterized by the reactivity of a serine residue in the active site of the enzyme. Elastase breaks down elastin, the specific protein of elastic fibers, and digests other proteins such as fibrin, hemoglobin and albumin. Three structurally related types of elastase, named elastases I, II and III (or protease E), have been identified, with several isoforms being secreted by the mammalian exocrine pancreas. Elastase has been confirmed to exist in the pancreas of most mammals, including humans, monkeys, cats, rabbits, etc. Elastase disclosed herein can be derived from any source, and can be produced by genetic engineering techniques. Non-limiting examples of elastases that can be used are human elastase I, human elastase II, and human elastase III.

In some embodiments, hyaluronidase, elastase, or combination thereof may be present in the composition from about 0.1 wt. % to about 10 wt. %, about 0.1 wt. % to about 9 wt. %, about 0.1 wt. % to about 8 wt. %, about 0.1 wt. % to about 7 wt. %, about 0.1 wt. % to about 6 wt. %, about 0.1 wt. % to about 5 wt. %, about 0.1 wt. % to about 4 wt. %, about 0.1 wt. % to about 3 wt. %, about 0.1 wt. % to about 3 wt. %, or about 0.1 wt. % to about 1 wt. %. Specific examples include about 0.1 wt. %, about 0.5 wt. %, about 1 wt. %, about 2 wt. %, about 5 wt. %, about 10 wt. %, and ranges between any two of these values. The weight percentages disclosed herein may be weight-to-weight or weight-to-volume percentages with respect to the total amount of the composition.

In embodiments, the composition may include pharmaceutically or cosmetically acceptable carriers, excipients, additives, as described herein. For example, in some embodiments, the composition can include one or more penetration or permeation enhancers in addition to or in place of the decoy molecule. Chemical permeation enhancers (CPEs) for transdermal drug delivery. CPEs are known in the art. Suitable CPEs may include, but are not limited to, ethylene-diamine tetra-acetic acid (EDTA), bile salt permeation enhancers, such as sodium deoxycholate, sodium taurocholate, sodium deoxycholate, sodium taurodiliydro fusidate, sodium dodecyl sulfate, sodium glycocholate, taurocholate, glycocholate, taurocheno-deoxycholate, taurodeoxycholate, deoxycholate, glycodeoxycholate, and ursodeoxycholate, fatty acid permeation enhancers, such as sodium caprate, sodium laurate, sodium caprylate, capric acid, lauric acid, and caprylic acid, acyl carnitines, such as palmitoyl carnitine, stearoyl carnitine, myristoyl carnitine, and lauroyl carnitine, and salicylates, such as sodium salicylate, 5-methoxy salicylate, and methyl salicylate. The amount of permeation enhancer included in the compositions may vary from about 0.1 wt % to about 40 wt %, depending on, for example, the active agent to be delivered, the nature of the permeation enhancer itself, and the dose of formulation to be administered. For example, the permeation enhancers can be included in the formulations in a total amount of about 0.1 wt. % to about 15 wt. %, about 2 wt. % to about 12 wt. %, about 4 wt. % to about 10 wt. %, about 4 wt. % to about 7 wt. %, about 4 wt. % to about 6 wt. %, about 4.5 wt. % to about 5.5 wt. %, about 4 wt. % to about 5 wt. %, or any range or individual concentration within these example ranges.

In some embodiments, the penetration or permeation enhancer may be a peptide or protein fragment. Such peptides and protein fragments are generally termed “skin penetrating peptides” (SPPs) or cell penetrating peptides (CPPs). SPPs may stabilize these structural proteins in the skin rather than denaturing them. For example, SPPs bind to keratin proteins through hydrogen bonds and weak electrostatic interactions and may operate as binding mediators between keratin and drug molecules. SPPs may also utilize pathways between corneocytes via diffusion of drug via gaps between cells as well as through lipid bilayers, without disruption. An example of a SPP is TD-1, which is known to loosen the desmosome-induced tight junctions between corneocytes with a change in the space between cells from about 30 nm to about 466 nm in 30 minutes from topical application. The cell gaps increase and then gradually are restored in 1 hour after treatment with TD-1. Various SPPs are known in the art and numerous peptides containing 9 to 19 amino acids have been shown to exhibit skin penetrating activity. Embodiments encompass all such peptides.

Nanoparticles and microparticles can be used to aid in the delivery of compositions described above. In some embodiments, the decoy and active agent may be encapsulated within the nanoparticles or microparticles or ionically associated with the nanoparticle or microparticles. In other embodiments, the active agent can be encapsulated in the nanoparticles or microparticles and the decoy may be combined and unassociated with the nanoparticles or microparticles or ionically associated with the nanoparticle or microparticles, and in further embodiments, a portion of the active agent, extracellular matrix component, or both the active agent and extracellular matrix component may be encapsulated in the nanoparticles or microparticles and a portion of the active agent, extracellular matrix component, or both the active agent and extracellular matrix component are not encapsulated by the nanoparticles or microparticles.

In some embodiments, the compositions may include lipid nanoparticles or microparticles. Such nanoparticles can be prepared by forming an emulsion of active agent, extracellular matrix component, or both active agent and extracellular matrix component may be dispersed or dissolved in a solvent, and this solution may be combined with glycerol and poloxomer to form an emulsion. The emulsion can be heated, cooled, and homogenized to produce microparticles or nanoparticles. In other embodiments, the compositions may include commercially available nanoparticles or microparticles such as, for example, hybrid polyamidoamine (PAMAM) dendrimer hydrogel/poly (lactic-co-glycolic acid) (PLGA) nanoparticles or microparticles (HDNP), chitosan (CS) nanoparticles or microparticles, thiolated chitosan nanoparticles or microparticles, calcium phosphate (CaP) nanoparticles or microparticles, poly (lactic-co-glycolic acid) copolymer (PLGA), poly (ethylene glycol)-block-poly(-caprolactone) nanopolymeric nanoparticles or microparticles, core/shell nanoparticles or microparticles composed of, for example, a lecithin liposome as the core and pluronic F 127 diacrylate (DA-PF 127), inorganically-coated retinoic acid (atRA) nanoparticles or microparticles, poly (lactic acid) (PLA) homopolymers and PEG-block-PLA copolymer nanoparticles or microparticles, PEG-block-PPG copolymers such as Pluronic, PEGylated liposome-protamine-hyaluronic acid nanoparticles or microparticles, polylactic acid/polylactic acid-polyethylene oxide (PLA/PLA-PEO) nanoparticles or microparticles, and the like and combinations thereof. In various embodiments, the nanoparticles may have a diameter of from about 2 to about 200 nanometers, about 5 to about 50 nanometers, or about 18 to about 22 nanometers, or any range or individual value encompassed by these ranges.

In various embodiments, such formulations may include the composition, i.e. active and decoy, encapsulated by the microparticle or nanoparticle. In other embodiments, one or the other of the active agent or decoy may be encapsulated by the microparticle or nanoparticle. In still other embodiments, a portion of the composition may be encapsulated by the microparticles or nanoparticles and another portion of the composition may not be encapsulated by the microparticles or nanoparticles. In each such embodiment, the active agent or decoy may be ionically or covalently associated with the microparticles or nanoparticles or portions thereof either within the microparticle or nanoparticles or on an outer surface of the microparticle or nanoparticle. In some embodiments, the compositions described above may be encapsulated, or at least partially encapsulated in liposomes. Liposomes are well known and commonly used in the pharmaceutical arts, and any type of liposome can be used in the compositions of embodiments. In some embodiments, the liposomes may be composed of phosphatidylcholine (PC) and other constituents such as cholesterol and lipid-conjugated hydrophilic polymers. In other embodiments, the liposomes may contain chitosan or may be coated in chitosan (i.e. chitosomes).

In particular embodiments, the compositions may include colloidal lipids. Such compositions may include colloidal polar lipids formed from one or more non-ionic polyethylene glycol derivatives of castor oil and/or hydrogenated castor oil such as, for example, PEG-30 castor oil, PEG-33 castor oil, PEG-36 castor oil, PEG-40 castor oil, PEG-30 hydrogenated castor oil and PEG-40 hydrogenated castor oil, an anionic purified polysaccharide such as Gellan Gum, one or more buffering agents such as, for example, boric acid, trimethamine, and, in some embodiments, one or more aqueous lubricants and one or more colloidal aqueous lubricants.

The liposomes or colloidal lipids may form particles about 1 nanometers to about 50 nanometers or about 6 nanometers to about 22 nanometers. The compositions of such embodiments may include about 0.1 w/v % to about 15 w/v % lipids.

In some embodiments, the compositions may further include pharmaceutical and/or cosmetically acceptable carries, excipients, diluents, fillers, disintegrants, desiccants, binders, lubricants, surfactants, hydrophobic vehicles, water soluble vehicles, emulsifiers, buffers, humectants, moisturizers, solubilizers, preservatives, colorants, plasticizers, carriers, or combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments. Any previously mentioned, carries, excipients, diluents, fillers, disintegrants, desiccants, binders, lubricants, surfactants, hydrophobic vehicles, water soluble vehicles, emulsifiers, buffers, humectants, moisturizers, solubilizers, preservatives, colorants, plasticizers, carriers, and combinations thereof may be incorporated into such compositions.

In some embodiments, the compositions may further include one or more additives selected from the group consisting of vitamins, cosmetic peptides, oil control agents, sensation modifying agents, skin lightening agents, hydrating compositions, a sunblock agent, a compound that absorbs or reflects UV photons, other skin care agent and combinations thereof.

In embodiments, the compositions may include vitamins, including, but not limited, to vitamin D, vitamin K, vitamin B (including niacinamide, nicotinic acid, C₁₋₁₈ nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

In embodiments, the compositions may include cosmetic peptides, including, but not limited, to di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (MATRIXYL®) palmitoyl-glycine-glutamine-proline-arginine (RIGIN®), these three being available from Sederma, France, and Cu-histidine-glycine-glycine (Cu-HGG, also known as IAMIN®), and naturally occurring and synthesized derivatives thereof, and combinations thereof. In various embodiments, the compositions may include about 1×10⁻⁷ wt. % to about 20 wt. %, about 1×10⁻⁶ wt. % to about 10 wt. %, and about 1×10⁻⁵ wt. % to about 5 wt. %, or any individual concentration or range of each peptide contained in the composition. Such cosmetic peptides may be dried or dehydrated to form a powder by the methods described above.

In embodiments, the compositions may include other skin care agents, including, but not limited to, retinol, steroids, sunblock, salicylate, minocycline, antifungals, peptides, antibodies, lidocaine, and the like and combinations thereof. In some embodiments, other skin care agents include N-acyl amino acid compounds including, for example, N-acyl phenylalanine,

N-acyl tyrosine, and the like, their isomers, including their D and L isomers, salts, derivatives, and mixtures thereof. An example of a suitable N-acyl amino acid is N-undecylenoyl-L-phenylalanine is commercially available under the trade name SEPIWHITE®. Other skin active agents include, but are not limited to, Lavandox, Thallasine 2, Argireline NP, Gatuline In-Tense and Gatuline Expression, Myoxinol LS 9736, Syn-ake, and Instensyl®, Sesaflash™, N-acetyl D-glucosamine, panthenol (for example, DL panthenol available from Alps Pharmaceutical Inc.), tocopheryl nicotinate, benzoyl peroxide, 3-hydroxy benzoic acid, flavonoids (for example, flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (for example, retinyl propionate), phytic acid, N-acetyl-L-cysteine, lipoic acid, tocopherol and its esters (for example, tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. Further skin care agents are disclosed in US Publication No. 2007/0020220A1, wherein the components/ingredients are incorporated herein by reference in their entirety.

In embodiments, the compositions may include skin lightening agents, such as ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and combinations thereof.

In some embodiments, the compositions may include sunblock agents, such as but not limited to para-aminobenzoic acid (PABA), PABA esters (glyceryl PABA, amyl dimethyl PABA and octyl dimethyl PABA), benzophenones (oxybenzone and sulisobenzone), cinnamates (octylmethoxy cinnamate and cinoxate), salicylates (homomethyl salicylate) anthranilates, TiO₂, avobenzone, bemotrizinol, bisoctrizole, 3-(4-methylbenzylidene)-camphor, cinoxate, diethylamino hydroxybenzoyl hexyl benzoate, dioxybenzone, drometrizole trisiloxane, ecamsule, ethylhexyl triazone, homosalate, menthyl anthranilate, octocrylene, octyl salicylate, iscotrizinol, isopentenyl-4-methoxycinnamate, octyl-dimethyl-p-aminobenzoic acid, octyl-methoxycinnamate, oxybenzone, polysilicone-15, trolamine salicylate, and ZnO. In some embodiments, any active agent disclosed herein can be combined with any of the sunblock agents disclosed herein or known in the art.

In embodiments, the composition may further include a sensation modifying agent selected from the group of a cooling agent, a warming agent, a relaxing or soothing agent, a stimulating or refreshing agent, and combinations thereof.

In embodiments, the cooling agent is selected from but not limited to menthol; an isomer of menthol, a menthol derivative; 4-methyl-3-(1-pyrrolidinyl)-2[5H]-furanone; WS-23, Icilin, Icilin Unilever Analog, 5 -methyl-4-(1-pyrrolidinyl)-3 -[2H] -furanone; 4,5 -dimethyl-3-(1-pyrrolidinyl)-2 [5H]-furanone; i sopulegol, 3-(1-menthoxy)propane-1,2-diol, 3-(1-menthoxy)-2-methylpropane-1,2-diol, p-menthane-2,3 -diol, p-menthane-3 , 8-diol, 6-isopropyl-9-methyl-1,4-dioxas-piro[4,5]decane-2-methanol, menthyl succinate and its alkaline earth metal salts, trim ethylcyclohexanol, N-ethyl-2-isopropyl-5 -m ethylcyclohexanecarb-oxamide, Japanese mint (Mentha arvensis) oil, peppermint oil, menthone, menthone glycerol ketal, menthyl lactate, 3-(1-menthoxy)ethan-l-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide and spearmint oil.

In embodiments, the warming agent is selected from but not limited to polyhydric alcohols, capsaicin, capsicum powder, a capsicum tincture, capsicum extract, capsaicin, hamamelis, homocapsaicin, homodihydrocapsaicin, nonanoyl vanillyl amide, nonanoic acid vanillyl ether, vanillyl alcohol alkyl ether derivatives, such as vanillyl ethyl ether, vanillyl butyl ether, vanillyl pentyl ether, and vanillyl hexyl ether, iso vanillyl alcohol alkyl ethers, ethyl vanillyl alcohol alkyl ethers, veratryl alcohol derivatives, substituted benzyl alcohol derivatives, substituted benzyl alcohol alkyl ethers, vanillin propylene glycol acetal, ethylvanillin propylene glycol acetal, ginger extract, ginger oil, gingerol and zingerone.

In embodiments, the relaxing or soothing agent is selected from but not limited to herb extracts, selected from the group consisting of aloe vera, alpha bisabolol, D-panthenol, allantoin, hamamelis, chamomile, yarrow; calendula, comfrey, witch hazel and other astringents, sea weed, and oat extracts; oils, selected from the group consisting of: almond oil, avocado oil, and comfrey; and essential oils, selected from the group consisting of: cardamone, eucalyptus, mentha piperita (peppermint), hyssop, and rosemary; waxy or unctuous substances selected from the group consisting of: lanolin or vaseline jelly, minerals, selected from the group consisting of: zinc oxide, calamine and selenium; vitamins, selected from the group consisting of: tocopheryl acetate (vitamin E), and pharmaceutical agents selected from the group consisting of: analgesics, anesthetics, anti-inflammatory agents, and antihistamines, and muscle relaxants; menthol, camphor, eugenol, eucalyptol, safrole, methyl salicylate, menthyl lactate, menthyl ethoxyacetate, menthone glycerinacetal, 3-1-methoxypropane-1,2-diol, ethyl 1-methyl carbonate, (1S,3S,4R)-p-menth-8-en-3-ol, methyl pyrrolidone carboxylate, N-substituted-p-menthane-3-carboxamides hamamelis extract and ginger oil.

In embodiments, the stimulating or refreshing agent is selected from but not limited to an alcohol, L-menthol, camphor, menthe oil, capsicum extract, capsaicin, benzyl nicotinate, salicylate, glycol salicylate, acetylcholine, serotonin, histamine, a prostaglandin, a neurotransmitter; a CNS stimulant, caffeine and quinine.

In embodiments, the compositions may include a second active agent. In embodiments, the second active agent is selected from analgesic agents, antifungal agents, antibacterial agents, anesthetic agents, anti-inflammatory agents, anti-rosacea agents, vasoconstrictors, anti-acne agents, anti-claudication agents, skin retexturing agents and steroids including, but not limited to, retinoids (retinol, retinal, retinoic acid, retinyl propionate), salicylates (acetyl salicylic acid, methyl salicylate, salicylic acid), benzoyl peroxide, minocycline, clindamycin hydrochloride, clindamycin phosphate, erythromycin, tetracycline, dicloxacillin, doxycycline, tretinoin, isotretinoin, adapalene, gabapentin, pregabalin, cyclosporine, tacrolimus (FK506), oxymetazoline, brimonidine, tetrahydrozoline, phenylephrine, clopidogrel, prasugrel, ticagrelor, ticlopidine, bimatoprost and other PGE2 inhibitors, tadalafil, clindamycin, cortisone, minoxidil, minoxidil sulfate, niacinamide, gabapentin, hydrocortisone, palmitoyl-KTTKS peptide, phenytoin, vitamin B12, cyclobenzaprine, anastrozole, lidocaine, minocycline, gentamicin sulfate, bimatoprost, minoxidil sulfate, clobetasol propionate, ascorbic acid, tranexamic acid, salicylic acid (sodium salicylate), hydroquinone, Renokin®, tolnaftate, clotrimazole, terbinafine, isotretinoin, tretinoin, kojic acid, prednisone, a sunscreen actives such as homosalate, octisalate, octocrylene, or avobenzone, hydrocortisone, lidocaine, ixekizumab taltz, aminolevulinic acid (ALA), baricitinib, tofacitinib, adalimumab, citronella oil, 3(N-butyl-N-acetyl)aminopropionic acid ethyl ester, sarecycline, D3 analogs, calcineurin inhibitors, mechlorethamine, immunization antigens, imiquimod, ibuprofen, celecoxib, diclofenac, sildenafil, ciclopirox, sarecycline, estrogen, conjugated estrogens (PREMARIN®), potassium hydroxide, podophyllin, cantharidin, imiquimod, nitric acid, oral cimetidine, 5-fluorouracil, bleomycin, DNCB, imiquimod, and trichloroacetic acid, bleomycin, 2,4-dinitrochlorobenzene, fluorouracil, silver nitrate, zinc sulfate, zinc oxide, bacitracin, chlortetracycline, neomycin, mupirocin, polymyxin B, cuprimyxin, furazolidone, gentamicin, lincomycin, cephalosporins, beta lactam antibiotics, lincomycin hydrochloride, tazarotene, vitamin A, acitretin, bexarotene, oxybutynin; vitamin D, vitamin C, vitamin B, vitamin E; sulfur; glucocorticosteroids, corticosteroids, triamcinolone, triamcinolone acetonide, betamethasone, betamethasone 1 7-valerate, betamethasone dipropionate, halcinonide, isoflupredone acetate, flumethasone, fluocinonide, mometasone, fluticasone, fluticasone propionate, prednisolone, becIomet(h)asone, hydrocortisone, cyproterone, drospirenone, estrogen, progestogen, tacrolimus, pimecrolimus, ursolic acid, betulinic acid, moronic acid, oleanolic acid, acyclovir, valaciclovir, famciclovir, penciclovir, docosanol, perillyl alcohol, and combinations thereof.

In embodiments, the compositions described herein may be formulated as aerosols, in which the composition is dissolved in a propellant such as dichlorodifluoromethane, trichlorofluoromethane, dichlorotetrafluoroethane, carbon dioxide, or other suitable gas, and a co-solvent such ethanol, acetone, hexadecyl alcohol, and the like and combinations thereof.

In embodiments, the compositions disclosed herein can be in the form of transdermal patches. The transdermal patches can be in any conventional form such as, for example, a strip, a gauze, a film, and the like. Patch material may be non woven or woven (e.g., gauze dressing). Layers may also be laminated during processing. It may be nonocclusive or occlusive, but the latter is preferred for backing layers. The patch is preferably hermetically sealed for storage (e.g., foil packaging). The patch can be held onto the skin and components of the patch can be held together using various adhesives. For example, the transdermal patch can be in the form of a band-aid type device, or it may be packaged in a small metal or plastic “cup”, which is strapped onto the appropriate site using an adhesive, tape, or an outer fabric or leather strap, similar to that worn as part of a watch. The entire patch may be disposable or may be refillable.

In embodiments, the compositions disclosed herein can be coated on bandages, mixed with bioadhesives, or included in dressings.

A wide variety of methods may be used for preparing the formulations described herein. Broadly speaking, the formulations may be prepared by combining together the components of the formulation, as described herein, at a temperature and for a time sufficient to provide a pharmaceutically acceptable composition. For example, in some embodiments, the components of the compositions may be dissolved, suspended, dispersed or otherwise mixed in a selected carrier or vehicle, at an effective concentration such that the condition to be treated is relieved or ameliorated.

The compositions of embodiments described above may enhance the strength of known topical active agents thereby reducing the necessary dosage required to achieve a therapeutically effective amount. For example, in some embodiments, the strength of a composition containing an active agent and decoy may be about equal to about 80% or 90% greater than the active agent delivered in a standard topical formulation. In other embodiments, the strength of a composition containing an active agent and decoy may be about equal to about 75% greater, about 1.0% to about 80% greater, about 1.0% to about 75% greater, about 1.0% to about 50% greater, about 1.0% to about 25% greater, about 2.0% to about 80% greater, about 2.0% to about 75% greater, about 2.0% to about 50% greater, about 2.0% to about 25% greater, about 5.0% to about 50% greater, about 5.0% to about 25% greater than the active agent delivered in a standard topical formulation. Thus, the compositions described herein may provide therapeutic equivalence of known topically administered active agents with that an administered dose that is equal to or at least about 75% less than a standard dose, equal to or about 50% less than a standard dose, equal to or about 25% less than a standard dose, equal to or about 10% less than a standard dose, about 1.0% to about 75% less than a standard dose, about 1.0% to about 50% less than a standard dose, about 1.0% to about 25% less than a standard dose, about 1.0% to about 10% less than a standard dose, about 2.0% to about 75% less than a standard dose, about 2.0% to about 50% less than a standard dose, about 2.0% to about 25% less than a standard dose, about 2.0% to about 10% less than a standard dose, or any range or individual value encompassed by these example ranges. The compositions disclosed herein may deliver the active agent more efficiently. That is, the effective amount of the active agent delivered at the site of administration is much more when compared to the delivery of the active agent without decoy.

Additional embodiments include methods for delivering an active agent. Such methods may include the step of applying a composition or formulation such as those described above including an active agent and decoy to a surface tissue of a subject. In other embodiments, the decoy may be applied to the surface tissue before topical administration of the active agent. In yet other embodiments, the active agent may be applied to a surface tissue followed by applying decoy to the surface tissue.

In some embodiments, the compositions may be delivered topically by, for example, applying the powdered compositions directly to skin of a patient in need for treatment at the location of the injury or disease. In other embodiments, the compositions may be applied locally to the skin of a patient before, for example, surgery, injections, or other medically necessary injury. In further embodiments, the compositions may be administered transdermally, percutaneously, or by microneedle injection. Administration can also be, for example, intravenous, intraperitoneal, subdermal, subcutaneous, intradermal, transcutaneous, intramuscular, oral, intra-joint, parenteral, intranasal, or by inhalation. Suitable sites of administration thus include, but are not limited to, the skin, bronchium, gastrointestinal tract, eye, buccal cavity, and ear.

In embodiments such as those described above, the composition can be applied to the surface tissue one or more times each day, and applying can be carried out for a period of at least 1 month, 2 months, 3 months, 4 months, 6 months, 8 months, 12 months, or indefinitely depending on the condition of the patient or disease or injury being treated. In some embodiments, the composition may be administered once, as needed, once daily, twice daily, three times a day, once a week, twice a week, every other week, every other day, or the like. A dosing cycle may include administration for about 1 week, about 2 weeks, about 3 weeks, about 4 weeks, about 5 weeks, about 6 weeks, about 7 weeks, about 8 weeks, about 9 weeks, or about 10 weeks. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

The methods of such embodiments can be used for treating nearly any condition. For example, the methods of embodiments can be used for treatment of pain arising from or associated with acute or chronic pain, somatic pain, visceral pain, inflammatory pain, neuropathy, arthritis, osteoarthritis, headache, and the like and combinations thereof. The pain may be local or systemic, and in some embodiments, the methods of the invention can be used for administering active agents for treating conditions that require transdermal delivery of the active agent. In particular, embodiments, the pain may be associated with chronic pain relief, cancer, motion sickness, chronic illnesses, acne, fungal or bacterial infections, skin cancer, abscesses, cellulitis, benign neoplasm, premalignancy, malignancy, warts, common warts, palmoplantar warts, flat warts, epidermodysplasia verruciformis related warts, anogenital warts, condyloma accuminatum; Herpesvirus related lesions including those induced by HHV-1 (HSV-1), HHV-2 (HSV-2), HHV-3 (varicella-zoster virus) e.g. chicken pox, Herpes zoster, shingles; Poxvirus induced lesions e.g. molluscum contagiosum, orf,; callus, cutaneous horns, corns, acrochordons, fibroepithelial polyps, prurigo nodularis, actinic keratoses, squamous cell carcinoma, squamous cell carcinoma in situ, keratoacanthoma, basal cell carcinoma, cutaneous lymphomas and benign lymphocytic infiltrates & hyperplasias of the skin, clear cell acanthoma, large cell acanthoma, epidermolytic acanthoma, porokeratosis, hyperkeratosis, lichenoid keratosis, acanthosis, acanthosis nigricans, confluent and reticulated papillomatosis, nevi, including e.g. dermal nevi, epidermal nevi, compound nevi, ILVEN (inflammatory linear verrucous epidermal nevi), nevus sebaceous, nevus comedonicus, and the like; acne, e.g. comedonal acne, inflammatory acne, papular acne, pustular acne, cystic acne; cysts, e.g. epidermoid cysts, milia, trichilemmal cysts, follicular cysts, proliferating cysts, dermoid cysts, pilonidal cysts, apocrine cysts, eccrine cysts, sebaceous cysts, mucous cysts, myxoid cysts, ganglion cysts, synovial cysts, vellus hair cysts, steatocystoma, hidrocystoma; adnexal neoplasms e.g. trichofolliculoma, fibrofolliculoma, perifollicular fibroma, trichodiscoma, nevus sebaceous, chondroid syringoma, trichoepithelioma, trichoblastoma, desmoplastic trichoepithelioma, pilomatricoma, pilomatrical carcinoma, trichilemmoma, trichilemmal carcinoma, tumor of the follicular infundibulum, trichoadenoma, proliferating pilar tumor, sebaceous hyperplasia, sebaceous adenoma, sebaceous epithelioma, sebaceous carcinoma, syringoma, poroma, hidradenoma, apocrine hidradenoma, spiradenoma, cylindroma, eccrine nevus (eccrine hamartoma), papillary adenoma, papillary adenocarcinoma; Benign melanocytic neoplasms e.g. ephelides, café-au-lait macules, Becker's melanosis, lentigines, solar lentigines, lentigo simplex, mucosal melanocytic lesions, Mongolian spots, Nevus of Ota, blue nevus, common acquired melanocytic nevi (nevocellular nevus, “moles”), congenital nevi, nevus spilus, recurrent nevi; vascular and perivascular neoplasms and reactive hyperplasias e.g., hemangiomas, cherry angiomas, hobnail hemangiomas (targeted hemosiderotic hemangiomas), tufted angiomas, hemangioendotheliomas, angiolymphoid hyperplasia with eosinophilia (ALHE), Glomus tumors (glomangiomas), hemangiopericytomas; cutaneous neural and neuroendocrine neoplasms e.g. neuromas, Schwannomas, neurofibromas, nerve sheath tumor, nerve sheath myxoma, neurothekeoma, granular cell tumor; fibrotic and fibrohistiocytic proliferations e.g. acrochordons, fibroepithelial polyps, fibromas, fibrous papules, angiofibromas, pearly penile papules, periungual fibromas, dermatofibromas, fibrokeratomas, sclerotic or pleomorphic fibromas, connective tissue nevi; cutaneous scars, hyperplasias, keloids, rosacea, cutaneous fungal, dermatophyte & mold infections, onychomycosis, hyperpigmentation, rhytides, psoriasis, malignant melanoma, seborrheic keratosis, seborrheic keratosis variants including e.g. dermatosis papulosis nigra, inverted follicular keratosis/keratoma warty dyskeratosis/warty dyskeratoma, acrokeratosis verruciformis, stucco keratosis, hyperhidrosis, pachyonychia congenita, and the like and combinations thereof.

The compositions disclosed herein can include additional active agents such as salicylic acid, potassium hydroxide, podophyllin, cantharidin, imiquimod, nitric acid, oral cimetidine, 5-fluorouracil, bleomycin, DNCB, imiquimod, and trichloroacetic acid, benzoyl peroxide, bleomycin, 2,4-dinitrochlorobenzene, fluorouracil, salicylic acid, silver nitrate, zinc sulfate, zinc oxide, clindamycin hydrochloride and clindamycin phosphate, erythromycin, tetracycline, dicloxacillin, doxycycline, minocycline, bacitracin, chlortetracycline, neomycin, mupirocin, polymyxin B, cuprimyxin, furazolidone, gentamicin, lincomycin, cephalosporins, beta lactam antibiotics, lincomycin hydrochloride, tazarotene, vitamin A, retinoic acid, tretinoin, isotretinoin, adapalene, retinol, acitretin, bexarotene, retinoids; oxybutynin; vitamin D, vitamin C, vitamin B, vitamin E; sulfur; glucocorticosteroids, corticosteroids, triamcinolone, triamcinolone acetonide, betamethasone, betamethasone 17-valerate, betamethasone dipropionate, halcinonide, isoflupredone acetate, flumethasone, fluocinonide, mometasone, fluticasone, fluticasone propionate, prednisolone, becIomet(h)asone, hydrocortisone, cyproterone, drospirenone, estrogen, progestogen, tacrolimus, pimecrolimus, ursolic acid, betulinic acid, moronic acid, oleanolic acid, acyclovir, valaciclovir, famciclovir, penciclovir, docosanol, perillyl alcohol, and combinations thereof.

As indicated above, a “surface tissue” includes any surface tissue such as, but not limited to, skin, mucosa, eyes, ears, inside the nose, inside the mouth, lips, urethral openings, vagina, anus, tongue, frenulum of tongue, hair, teeth, bone, and the like. Accordingly, administration of the compositions can be to any surface tissue, including skin, mucosa, eyes, ears, inside the nose, inside the mouth, lips, urethral openings, vaginal, anus, tongue, frenulum of tongue, hair, teeth, bone, and the like.

In some embodiments, the methods may include a variety of additional steps including, for example, cleaning the surface tissue at the site of applying, and the like. In some embodiments, the methods may further include ablation of the tissue surface before, during or after administration of the compositions described herein. In embodiments, tissue surface ablation may include electromagnetic radiation, laser, dermabrasion, chemical peel, ultrasound, heating, cooling, or by a needle. In certain embodiments, the tissue surface is ablated with abrasion. Abrasion of the outer layer or epidermis of the skin (dermabrasion) is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin. In some embodiments, the tissue surface is ablated with microdermabrasion. Microdermabrasion refers generally to a procedure in which the surface of the skin is removed due to mechanical rubbing by a handpiece emitting a stream of sand or grit. For example, a handpiece can be used to direct an air flow containing tiny crystals of aluminum oxide, sodium chloride, or sodium bicarbonate. The momentum of the grit tends to wear away two to three cell layers of the skin with each pass of the handpiece. Alternatively, new “crystal-free” microdermabrasion techniques utilize a diamond-tipped handpiece without a stream of grit.

In embodiments, the methods may further include photodynamic therapy before, during or after administration of the compositions described herein. Photodynamic therapy is a minimally invasive two-step medical procedure that uses photoactivatable drugs called photosensitizers to treat a range of diseases. First, a photosensitizer is administered and, once it has permeated the target tissue, the photosensitizer is then activated by exposure to a dose of electromagnetic (usually light) radiation at a particular wavelength. The compositions disclosed herein may contain a photosensitizer. In embodiments, any suitable photosensitizing agent or mixture of agents may be used herein. Generally, these will absorb radiation in the range of from about 380 nm to about 900 nm. As used herein, “photosensitizer” or “photosensitizing agent” preferably means a chemical compound which, when contacted by radiation of a certain wavelength, forms singlet oxygen or thermal energy. Non-limiting examples of photosensitizers include aminolevulinic acid esters, porphyrins, porphyrin derivatives, bacteriochlorins, isobacteriochlorins, phthalocyanine, naphthalocyanines, pyropheophorbides, sapphyrins, texaphyrins, tetrahydrochlorins, purpurins, porphycenes, phenothiaziniums, and metal complexes such as, but not limited to, tin, aluminum, zinc, lutetium, and tin ethyl etiopurpurin (SnET2), and combinations thereof.

1. A composition comprising:
a powdered active agent; and
a powdered penetration enhancer.

2. The composition of claim 1, wherein the penetration enhancer is selected from the group consisting of alcohols, glycols, fatty acids, fatty esters, fatty ethers, occlusive agents, surface active agents, dimethyl amino propionic acid derivatives, terpenes, decoy molecules, sulfoxides, cyclic ethers, amides, amines, and the like and combinations thereof

3. The composition of claim 1, wherein the penetration enhancer is selected from the group consisting of hyaluronic acid, collagen, fibronectin, elastin, lectin, fragments thereof, and combinations thereof.

4. The compositions of claim 1, wherein the penetration enhancer is dehydrated or lyophilized.

5. The composition of claim 1, wherein the penetration enhancers has an average molecular weight of about 2,000 Da to about 100,000 Da.

6. The composition of claim 1, wherein the powdered active agent and powdered penetration enhancer each individually have an average particle size of about 0.01 μm to about 100 μm.

7. The composition of claim 1, wherein the active agent is selected from the group consisting of baclofen, vigabatrin, gabapentin, pregabalin, y-amino-phosphinic acid derivatives, and combinations thereof.

8. The composition of claim 1, wherein the composition is substantially free of water.

9. The composition of claim 1, comprising about 1 wt. % to about 95 wt. % active agent.

10. The composition of claim 1, comprising about 0.1 wt. % to about 10 wt. % penetration enhancer.

11. The composition of claim 1, wherein penetration enhancer is selected from the group consisting of chemical permeation enhancers (CPEs), non-ionizable glycol ethers, known peptides or protein fragments, microparticles or nanoparticles, and combinations thereof.

12. The composition of claim 1, further comprising a stabilizer is selected from the group consisting of alkanols, disodium EDTA (ethylenediamine tetraacetate), EDTA salts, EDTA fatty acid conjugates, isothiazolinone, parabens, methylparaben and propylparaben, propylene glycols, sorbates, urea derivatives, diazolidinyl urea, and combinations thereof

13. The composition of claim 1, further comprising a carrier, excipient, diluent, filler, disintegrant, desiccant, binder, lubricant, surfactant, hydrophobic vehicle, water soluble vehicle, emulsifier, buffer, humectant, moisturizer, solubilizer, preservative, colorant, plasticizer, or combinations thereof.

14. A method of treating a subject in need treatment comprising topically administering to a surface tissue of the subject a composition comprising a powdered active agent, and a powdered penetration enhancer.

15. The method of claim 14, wherein the powdered active agent is selected from the group consisting of baclofen, vigabatrin, gabapentin, pregabalin, y-amino-phosphinic acid derivatives, and combinations thereof.

16. The method of claim 14, wherein the penetration enhancer is selected from the group consisting of alcohols, glycols, fatty acids, fatty esters, fatty ethers, occlusive agents, surface active agents, dimethyl amino propionic acid derivatives, terpenes, decoy molecules, sulfoxides, cyclic ethers, amides, amines, and the like and combinations thereof

17. The method of claim 14, wherein the penetration enhancer is selected from the group consisting of hyaluronic acid, collagen, fibronectin, elastin, lectin, fragments thereof, and combinations thereof.

18. The method of claim 14, wherein the composition is substantially free of water.

19. The method of claim 14, wherein the powdered active agent and powdered penetration enhancer each individually have an average particle size of about 0.01 μm to about 100 μm.

20. The method of claim 14, wherein the composition further comprises powdered carries, excipients, diluents, fillers, disintegrants, desiccants, binders, lubricants, surfactants, hydrophobic vehicles, emulsifiers, buffers, humectants, moisturizers, solubilizers, preservatives, colorants, plasticizers, carriers, or combinations thereof.
